(19) United States
(12) Patent Application Publication
(10) Pub. No.: US 2024/0032965 A1
(43) Pub. Date: Feb. 1, 2024

(54) METHODS AND SYSTEMS FOR TREATMENT OF INTERVERTEBRAL DISC BY INTRADISCAL MICRO-NEEDLE INSERTION

(71) Applicant: DiscGenics, Inc., Salt Lake City, UT (US)

(72) Inventors: Kevin T. Foley, Salt Lake City, UT (US); Lara Ionescu Silverman, Salt Lake City, UT (US)

(21) Appl. No.: 18/254,120

(22) PCT Filed: Nov. 24, 2021
(86) PCT No.: PCT/US2021/060769

§ 371 Date: May 23, 2023

Related U.S. Application Data
(60) Provisional application No. 63/117,907, filed on Nov. 24, 2020.

(51) Int. Cl. A61B 17/34; A61F 2/46

(52) U.S. Cl. CPC A61B 17/3472; A61F 2/4601

(57) ABSTRACT

Disclosed herein are methods and systems for treating an intervertebral disc in a subject in need thereof. In some embodiments, the subject's intervertebral disc has a reduced disc height due to degeneration, damage, and/or disease. In other embodiments, the subject is at risk of losing height in at least one intervertebral disc. In most embodiments, the disclosed methods comprise inserting a needle into the treated disc a first time and a second time. In many embodiments the first and second time may be separated by a period of days or weeks.

### CROSS REFERENCE TO RELATED APPLICATIONS

This application claims priority to U.S. Provisional Patent Application No. 63/117,907, filed Nov. 24, 2020, entitled “METHODS AND SYSTEMS FOR TREATMENT OF INTERVERTEBRAL DISC BY INTRADISCAL MICRO-NEEDLE INSERTION,” which is incorporated by reference herein, in the entirety and for all purposes.

### FIELD

The disclosed processes, methods, and systems are directed to treatment of intervertebral disc degeneration, damage, and/or disease.

### BACKGROUND

Low back pain (LBP) is a serious medical condition affecting about 12-30% of U.S. adults. Outside the U.S. LBP is leading cause of disability. LBP can be attributed to many potential factors, however, as discussed below, disc degeneration (DD) is caused by the progressive breakdown of the intervertebral disc. This breakdown results in abnormal tissue mechanics and inflammation within the disc environment. This, in turn, may lead to compression or shrinkage of the intervertebral disc height, which is correlated with increased pain.

Current treatment options for patients with DD are limited, and largely unsuccessful in alleviating pain and/or improving disability. Patients that fail to improve often look for alternative sources of relief. Oftentimes, this means turning to prescription opioids for pain management in a time when opioid usage, addiction and drug-related deaths are at an all-time high. In fact, LBP is the most common non-cancer reason for opioid prescriptions in the US.

What is needed is an alternative therapy for treating disc degeneration that can restore disc height, alleviate pain, and is accessible to the large numbers of people suffering from LBP.

### SUMMARY

Disclosed herein are methods for treating intervertebral disc, comprising steps of inserting a first needle into an untreated intervertebral disc of a subject at least one time, removing the needle to create a partially treated intervertebral disc, and inserting a second needle into the partially treated intervertebral disc at least one time and then removing the second needle, thereby treating the intervertebral disc. In some embodiments, there is a period of greater than 7 days between inserting the first needle and inserting the second needle, and/or the first or second needle may be inserted into the disc and removed two or more times. In various embodiments, the first or second needle may have a gauge between about 18 g and 30 g, and/or may have a length between about 10 mm and 100 mm. The first or second needle may be inserted into the untreated or partially treated intervertebral disc to a depth of at 2 mm or more, for example into the nucleus pulposus at least one time. The first and/or second needle may be solid or hollow, such as where the needle comprises a lumen for delivery of one or more fluids, for example delivery into the nucleus pulposus of the disc. In these embodiments, the composition may comprise one or more of a compound, a nutrient, a cell, and combinations thereof. The disclosed methods may aid in increasing disc height, for example by resulting in a disc height that is 105%, or more, of the disc height of the untreated disc, for example 105% to 200% the disc height of the untreated disc.

Also disclosed are systems for increasing the height of an intervertebral disc in a subject in need thereof, the systems comprising: a syringe, a needle, and an intervertebral disc visualizing device, and optionally a device or system for measuring the height of an intervertebral. In some embodiments, the intervertebral disc visualizing device may be a fluoroscope, or other device known to those of skill in the art. In various embodiments, the needle may have a gauge between 18 g and 30 g, and/or a length between about 10 mm and 100 mm. The needle may be hollow or solid.

Also disclosed, are methods for increasing intervertebral disc height in a subject, comprising: inserting a first needle into an untreated intervertebral disc of the subject at least one time; removing the needle from the untreated intervertebral disc to create a partially treated intervertebral disc, wherein the needle may be inserted into the nucleus pulposus at least one time; waiting a duration of greater than 14 days and inserting a second needle into the partially treated intervertebral disc at least one time and removing the second needle from the partially treated intervertebral disc, wherein the second needle is inserted into the nucleus pulposus at least one time; waiting a duration of greater than 7 days, and thereby increasing the intervertebral disc height of the treated intervertebral disc.

### BRIEF DESCRIPTION OF THE DRAWINGS

FIG. 1 is a graph showing disc height as a percentage of pre-injury disc height over an 8-week period. Treatments were administered at 2- and 4-weeks post injury.

FIG. 2 shows two bar graphs of, respectively, disc height at Week 8 (top), and the percent change in disc height from Week 2 to Week 8 (bottom).

FIG. 3 are micrographs of disc tissue from un-injured (left) and injured+treated (right) subjects. At right, are micrographs of disc tissue after a single treatment (top) and repeat treatment (bottom).

### DETAILED DESCRIPTION

Disclosed herein are methods and systems for treatment of intervertebral disc injury, disease, and/or degeneration. The disclosed systems and methods may improve disc height and correlate with ameliorating or reducing pain in subjects having lower back pain and/or reduced disc height due to trauma, disease, and/or degeneration. In many embodiments, the disclosed methods and systems may result in increasing disc height by greater than 5%, 6%, 7%, 8%, 9%, 10%, 11%, 12%, 13%, 14%, 15%, 16%, 17%, 18%, 19%, 20%, 25%, 30%, 35%, 40%, or 45%, and less than about 50%, 45%, 35%, 30%, 25%, 24%, 23%, 22%, 21%, 20%, 19%, 18%, 17%, 16%, 15%, 14%, 13%, 12%, 11%, 10%, 9%, 8%, 7%, 6%, or 5% after a treatment with the method or system.

Ameliorate may refer to any improvement of a disease state (for example lower back pain, degenerative disc disease, damaged intervertebral disc) of a patient, by the administration of one or more treatments and/or compositions, according to the present disclosure, to such patient or subject in need thereof. Such an improvement may be seen as a slowing down the progression or stopping the progression of the disease of the patient, and/or as a decrease in severity of disease symptoms, an increase in frequency or duration of disease symptom-free periods or a prevention of impairment or disability due to the disease.

“Subject in need”, “patient” or those “in need of treatment” include those already with existing disease (i.e. degenerative disc disease, intervertebral disc injury, lower back pain, etc.), as well as those at risk of the disease. The terms also include human and other mammalian subjects that receive either prophylactic or therapeutic treatments as disclosed herein.

The term “mammal” includes, but is not limited to, humans, rabbits, mice, rats, guinea pigs, monkeys, dogs, cats, horses, cows, pigs, and sheep.

The terms “treat”, “treating” and “treatment” refer to eliminating, reducing, suppressing, or ameliorating, either temporarily or permanently, either partially or completely, a clinical symptom, manifestation or progression of an event, disease or condition associated with the intervertebral disc disorders and diseases described herein. As is recognized in the pertinent field, methods and drugs employed as therapies may reduce the severity of a given disease state but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, increasing intervertebral disc height, reducing back pain, etc. and/or reducing the number or severity of associated symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic treatment in an amount, duration, and repetition sufficient to induce a sustained improvement over pre-existing conditions, or a baseline indicator that reflects the severity of the particular disorder.

A treatment, as used herein may include one or more needle insertions into an intervertebral disc during a single physician, or qualified medical professional, visit or appointment. In many embodiments, the disclosed patient or subject's treatments, visits, or appointments may vary from two to five or more, for example more than 2, 3, 4, 5, 6, 7, 8, or 9. In many embodiments, the treatments/visits/appointments may be greater than a day apart, for example, where they are a day apart, the first treatment may be on Day 0 and the second may be on Day 1. Where treatments are two days apart, the first may be on Day 0 and the second on Day 2. In some embodiments, the treatments may be from 2 days to eight weeks apart, for example greater than 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 3 weeks, 4 weeks, 5 weeks, 6 weeks or 7 weeks apart, and less than about 8 weeks, 7 weeks, 6 weeks, 5 weeks, 4 weeks, 3 weeks, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, or 2 days apart.

As noted above, the disclosed methods and systems may help increase disc height that has been degraded or reduced by disease, injury, or other condition. In many embodiments, disc height may be increased from Day 0—i.e. the day of the first treatment. In many embodiments, an initial disc height is measured at or before the first treatment. The increase in disc height may be from about 10% to about 100%—that is the disc height may be 110% of the initial disc height to about 200% or 2-fold the initial disc height. In many embodiments, the increase may be more than about 10%, 15% 20%, 25%, 30%, 35%, 40%, 45%, 50%, 55%, 60%, 65%, 70%, 75%, 80%, 85%, 90%, or 95% the initial disc height and less than about 100%, 95%, 90%, 85%, 80%, 75%, 70%, 65%, 60%, 55%, 50%, 45%, 40%, 35%, 30%, 25%, 20%, 15%, 10%, 5%, or 1% of initial disc height.

The intervertebral disc height may be measured by various methods known to those of skill in the art. In many embodiments, height is measured using magnetic resonance imaging (MRI), computed tomography (CT), and/or radiograph.

The disclosed methods and systems may involve at least two insertions of a needle into the damaged disc. In many embodiments, a needle may be inserted 2, 3, 4, 5, 6, 7, 8, 9, 10, or more times in one visit. In some embodiments, the subject may return more than twice to receive treatment by the disclosed methods and systems.

The disclosed methods and systems may be practiced with needles of various sizes. In one embodiment, the needle may from about 10 g to about 30 g, for example about 25 g to about 30 g. For example, greater than 14 g, 15 g, 16 g, 17 g, 18 g, 19 g, 20 g, 21 g, 22 g, 23 g, 24 g, 25 g, 26 g, 27 g, 28 g, 29 g, or 30 g and less than about 31 g, 30 g, 29 g, 28 g, 27 g, 26 g, 25 g, 24 g, 23 g, 22 g, 21 g, 20 g, 19 g, 18 g, 17 g, 16 g, or 15 g. The needle may be of various lengths, for example from about to about 100 mm, for example greater than 10 mm, 15 mm, 20 mm, 25 mm, 30 mm, 35 mm, 45 mm, 50 mm, 55 mm, 60 mm, 65 mm, 70 mm, 75 mm, 80 mm, 85 mm, 90 mm, 95 mm, or 100 mm and less than about 150, 100 mm, 95 mm, 90 mm, 85 mm, 80 mm, 75 mm, 70 mm, 65 mm, 60 mm, 55 mm, 50 mm, 45 mm, 40 mm, 35 mm, 30 mm, 25 mm, 20 mm, or 15 mm. The needles disclosed herein may be hollow, or, if no composition is injected into the intervertebral disc, the needle may be closed.

The needle may contact the disc at one or more positions of the intervertebral disc (e.g. lateral, anterior, posterior) and may penetrate to various depths. In many embodiments, the needle may be inserted into the disc at 2 or more positions, for example more than one, two, three, four, five, six, seven, eight, nine, or ten positions, and less than 15, 10, nine, eight, seven, six, five, four, three, or two positions.

The needle insertions may be of various depths into the intervertebral disc (measured from the surface where the needle penetrates the annulus pulposus to the distal tip of the needle) and the needles may have sufficient corresponding length. In most embodiments, the needle tip may traverse the annulus pulposus and enter the nucleus pulposus. In many embodiments, the depth of insertion into the disc may be more than 2 mm, 3 mm, 4 mm, 5 mm, 6 mm, 7 mm, 8 mm, 9 mm, 11 mm, 12 mm, 13 mm, 14 mm, 15 mm, 16 mm, 17 mm, 18 mm, 19 mm, 20 mm, 21 mm, 22 mm, 23 mm, or 24 mm and less than about 25 mm, 24 mm, 23 mm, 22 mm, 21 mm, 20 mm, 19 mm, 18 v 17 mm, 16 mm, 15 mm, 14 mm, 13 mm, 12 mm, 11 mm, 10 mm, 9 mm, 8 mm, 7 mm, 6 mm, 4 mm, 3 mm, or 2 mm. The depth of penetration may vary depending upon the identity of the intervertebral disc (e.g. cervical, thoracic, lumbar), the age of the subject, and the position of entry of the needle.

The disclosed methods and systems may include delivery of a composition to the intervertebral disc. In these embodiments, the needle may be hollow to aid in delivery, and the needle may be inserted through the annulus pulposus into the nucleus pulposus tissue. In some embodiments, the composition may include one or more cell or non-cell compositions. Non-cell compositions may include one or more nutrients, compounds, or additives that may benefit, support, or enhance cell growth, for example growth of nucleus pulposus cells.

The disclosed methods and systems may be used to treat existing, or prevent development of, disc degeneration in a subject.

### EXAMPLES

### Example 1

Test rabbits were subjected to surgery to injure the disc 2 weeks prior to Day 1 of the Test Phase. An approximate 8-10 cm longitudinal incision was made on the lateral abdomen between the iliac crest and the last rib of the subject rabbit. The lumbar vertebral discs were accessed via retroperitoneal approach incising abdominal muscle layers but not entering the peritoneum. Blunt dissection was used to separate the peritoneum and hypaxial muscles to expose the ventral vertebral body. If the peritoneum was penetrated the procedure was continued as planned but abdominal organs and vessels were gently moved to visualize the vertebrae.

An 18-gauge needle was inserted approximately 5 mm into the lumbar vertebral discs of interest to create three disc injuries at L3/L4, L4/L5 and L5/L6, respectively. The muscle and skin was closed in two layers using appropriate absorbable suture (i.e. 3-0 Vicryl).

Surgery was performed using aseptic technique. Fluoroscopic imaging was used to help visualize the disc and vertebrae during the procedure.

For treatment, a Hamilton glass syringe and a 25-gauge needle was used. Specifically, at 2 weeks post-injury, subject rabbits were injected with: cell composition (labelled Cells), composition (labelled Composition), or nothing (labelled Insertion). Needles were inserted approximately 5 mm into the previously injured lumbar vertebral discs (i.e. L3/L4, L4/L5 and L5/L6). Treatment was repeated at 4 weeks post-injury.

Disc height was measured prior to injury, at 2 weeks, 3, weeks, 4 weeks, 6 weeks, and 8 weeks.

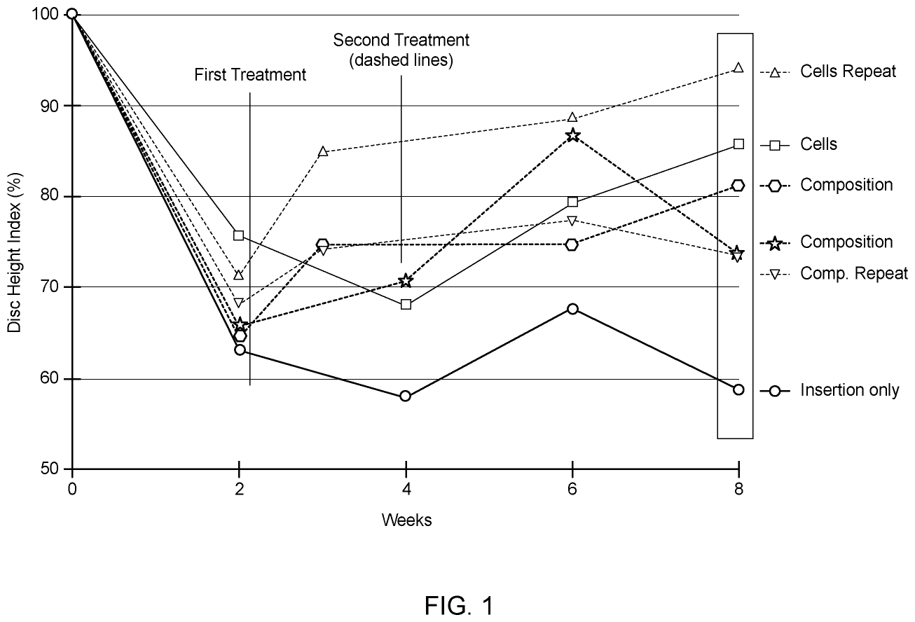

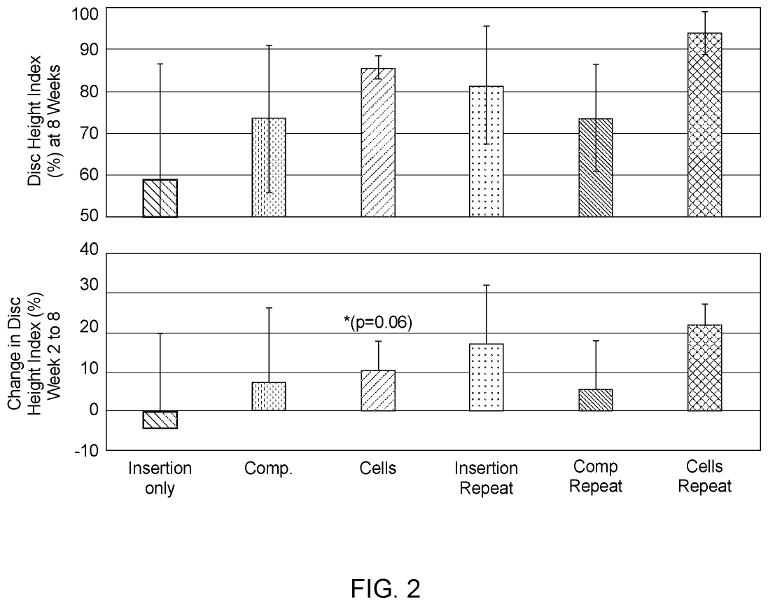

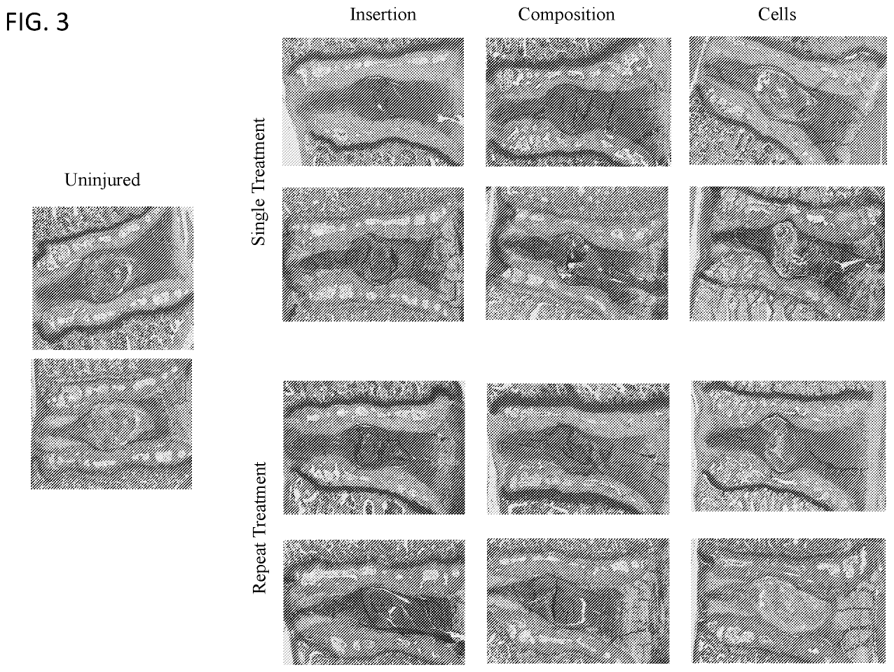

FIGS. 1-3 show results from these experiments. FIG. 1 is a graph showing disc height throughout the 8-week experiment, as a percentage of the measured disc height one day (Day 0) before injury (Day 1). FIG. 2 presents bar graphs showing disc height at 8 weeks (top), post injury, and change in disc height from week 2 to week 8 (bottom), respectively. Micrographs of discs before treatment (left) and after treatment (right) are shown in FIG. 3. FIG. 3 also presents micrographs of discs treated once (top right) and twice (bottom right).

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

1. A method for treating an intervertebral disc in a subject, comprising:
inserting a first needle into an untreated intervertebral disc of the subject at least one time,
removing the first needle from the untreated intervertebral disc to create a partially treated intervertebral disc,
inserting a second needle into the partially treated intervertebral disc at least one time,
removing the second needle from the partially treated intervertebral disc, and thereby
treating the intervertebral disc to create a treated intervertebral disc.

2. The method of claim 1, wherein between inserting the first needle and the second needle is a period of greater than 7 days.

3. The method of claim 1, wherein the first needle is inserted and removed twice from the untreated intervertebral disc, and the second needle is inserted and removed twice from the partially treated intervertebral disc.

4. The method of claim 3, wherein the first or second needle has a gauge between about 18 g and 30 g.

5. The method of claim 4, wherein the first or second needle has a length between about 10 mm and 100 mm.

6. The method of claim 4, wherein the first or second needle is inserted into the intervertebral disc at least 2 mm.

7. The method of claim 5, wherein the first or second needle is inserted into the nucleus pulposus at least one time.

8. The method claim 1, wherein the first needle and the second needle are hollow and comprises a lumen for delivery of one or more fluids.

9. The method of claim 8, wherein a fluid composition is delivered through the needle lumen into the nucleus pulposus of the subject.

10. The method of claim 9, wherein the composition comprises a non-cell composition comprising one or more of a compound, a nutrient, and an additive that benefit, support, or enhance nucleus pulposus cell growth.

11. The method of claim 3, wherein the first needle or second needle is solid.

12. The method of claim 1, wherein the treated intervertebral disc has a disc height that is at least 105% the disc height of the untreated disc.

13. The method of claim 7, wherein the treated intervertebral disc has a disc height that is between 105% and 200% the disc height of the untreated disc.

14. A system for increasing the height of an intervertebral disc in a subject, comprising:
a syringe;
a needle; and
an intervertebral disc visualizing device.

15. The system of claim 14, further including a device for measuring disc height.

16. The system of claim 15, wherein the intervertebral disc visualizing device is a fluoroscope.

17. The system of claim 16, wherein the needle has a gauge between 18 g and 30 g.

18. The system of claim 17, wherein the needle has length between about 10 mm and 100 mm.

19. The system of claim 18, wherein the needle is hollow.

20. A method for increasing intervertebral disc height in a subject, comprising:
inserting a first needle into an untreated intervertebral disc of the subject at least one time;
removing the first needle from the untreated intervertebral disc to create a partially treated intervertebral disc, wherein the needle is inserted into the nucleus pulposus at least one time;
waiting a duration of greater than 14 days;
inserting a second needle into the partially treated intervertebral disc at least one time;
removing the second needle from the partially treated intervertebral disc, wherein the needle is inserted into the nucleus pulposus at least one time;
waiting a duration of greater than 7 days, and thereby
increasing the intervertebral disc height of the treated intervertebral disc.
